(19) United States
(12) Patent Application Publication
(10) Pub. No.: US 2009/0123596 A1
(43) Pub. Date: May 14, 2009

(54) Gastro-Activated Dietary Fibers

(75) Inventor: Olav Gaserod, Steinberg (NO)

Correspondence Address: PATENT ADMINISTRATOR;FMC CORPORATION, 1735 MARKET STREET, PHILADELPHIA, PA, 19103, US

(73) Assignee: FMC Biopolymer AS, Drammen (NO)

(21) Appl. No.: 12/305,469

(22) PCT Filed: Jun. 18, 2007
(86) PCT No.: PCT/IB2007/001627

§ 371 Date: Dec. 18, 2008

Related U.S. Application Data
(60) Provisional application No. 60/815,401, filed on Jun. 21, 2006.
(60) Provisional application No. 60/941,177, filed on May 31, 2007.

(51) Int. Cl. A23L 1/308; A23L 1/0532; A23L 1/09; A23L 1/0524; C07H 3/00

(52) U.S. Cl. 426/2; 426/615; 426/74; 536/123.1

(57) ABSTRACT

A gastro-activated dietary fiber comprising an insoluble polysaccharide salt and use of the gastro-activated dietary fiber in ingestible products. The gastro-activated dietary fibers may include salts of biopolymers such as alginates, carrageenans and pectins that are insoluble in water and exhibit minimal or no swelling in water. The water swelling ratio of the gastro-activated dietary fiber may 15 or less. The gastro-activation of the dietary fiber may be induced by exposure to an acidic pH of 3 or less for at least 15 minutes. Ingestible compositions containing the gastro-activated dietary fiber solubilize and provide a viscosity increase in the digestive tract as a result of activation of the dietary fiber by natural excretions of the small intestine which induce gelation of the dietary fiber. Beneficial health effects are potentially obtained by the provision of dietary fiber in this manner, without the usual disadvantages of reduced palatability and poor mouthfeel that are associated with soluble fiber-containing ingestible compositions.

### FIELD OF THE INVENTION

The invention is directed towards gastro-activated polysaccharide-based dietary fibers and their use in ingestible products.

### BACKGROUND

Polysaccharide salts such as alginates are widely used in food applications as a gelling, film forming, stabilizing and/or thickening agent to modify the sensory or functional properties of the food. Polysaccharide salts are also a source of dietary fiber. Increased dietary fiber is a significant trend in the “health and wellness” food market segment. The intake of dietary fibers is generally considered to be beneficial to both cardiovascular and colonic health. The action of dietary fibers on the digestive tract includes reduction of intestinal absorption rates, reduction of colonic luminal toxicity, alteration of colonic microflora, and improvements in the colonic mucosal barrier (Brownlee, et al., “Alginate as a Source of Dietary Fiber”, Critical Reviews in Food Science and Nutrition, Vol. 45, pp 497 to 510, 2005).

Sodium alginate is the most commonly used alginate in foods, and it is regarded as a soluble dietary fiber. Dietary fiber is not degraded in the small intestine, binds water and increases the viscosity of the intestinal fluids. Functional properties and desired dietary fiber properties, however, can present difficulties for the processing of the food and/or have negative impact on the food palatability (Williams et al., “Inclusion of Guar Gum and Alginate into a Crispy Bar Improves Postprandial Glycemia”, Humans J. Nutr., Vol. 134 pp 886 to 889, 2004). For instance a high-fiber biscuit that swells and dries out your mouth when eaten or a highly viscous beverage do not appeal to most consumers. Furthermore, since significant amounts of fiber are needed in a product to truly give a beneficial effect for the consumer, it is difficult to formulate functional foods using a soluble dietary fiber such as sodium alginate.

U.S. Pat. No. 3,395,021 (Glicksman), issued Jul. 30, 1968, discloses a thickening agent for beverages comprising 0.4 to 3.5% of a water soluble gum and 0.1 to 1.5% of a water-swellable gum, where the water-swellable gum provides a pulpy mouthfeel.

U.S. Pat. No. 5,283,076 (Kazuyuki), issued Feb. 1, 1994, and U.S. Pat. No. 5,324,526 (Kazuyuki), issued Jun. 28, 1994, disclose drinkable beverages containing 1 to 50% of a water soluble algin with a decreased molecular weight in the range of 10,000 to 900,000.

U.S. patent application publication no. 2003/0124170 A1 (Gallaher), published Jul. 3, 2003, discloses administration of edible viscous polysaccharides that are water soluble, non-nutritive, and indigestible for reducing the body fat and/or leptin levels in mammals. The polysaccharides increase the viscosity of the water portion of the intestinal contents.

Pelkman, C. L., et al., “Consumption of a novel calcium-alginate beverage reduced energy intake in non-dieting, overweight and obese women” The FASEB Journal, Vol. 20, A1002 (2006) discloses enhanced satiety and reduced caloric intake by ingestion of a soluble fiber drink and a soluble calcium supplement which together form a cross-linked gel structure in the stomach.

U.S. patent application publication nos. US 2002/0193344 A1, US 2007/0082027 A1, US 2007/0082029 A1, US 2007/0082030 A1, US 2007/0082085 A1, US 2007/0082108 A1, US 2007/0082107 A1 and US 2007/0082114 A1 relate to methods of inducing enhanced satiety by administration of soluble anionic polysaccharides in combination with gelling cations.

U.S. patent application publication no. US 2006/0159823 A1 discloses self-gelling alginate systems comprising soluble alginate in combination with insoluble alginate/gelling ion particles.

U.S. patent application publication no. US 2006/0099324 A1 (Aurio), discloses the use of edible proteins to modulate the viscosity of food products such as beverages containing soluble polysaccharide fiber.

Protanal® TXF 200 calcium alginate (FMC Corporation) has been used in some bakery cream products, but not for the purpose of providing a gastro-activated dietary fiber in the small intestine.

In view of the foregoing, there remains a need in the art for gelling systems which provide a satiety effect and which may alleviate one or more of the disadvantages of poor taste and mouthfeel, compatibility with various products and gelation in the mouth.

### SUMMARY OF THE INVENTION

In one aspect, the invention is directed towards a gastro-activated dietary fiber comprising an insoluble polysaccharide salt and use of the gastro-activated dietary fiber in ingestible products. The gastro-activated dietary fibers may include salts of biopolymers such as alginates, carrageenans and pectins that are insoluble in water and exhibit minimal or no swelling in water. In one embodiment, the water swelling ratio of the gastro-activated dietary fiber is 15 or less, alternatively, 10 or less, or may be 5 or less, when measured in water after 24 hours. The gastro-activation of the dietary fiber to develop a viscosity increase may be induced by exposure to an acidic pH of 3 or less for at least 15 minutes and solubilizing ions.

In another aspect, the polysaccharide salt is a calcium alginate which has a Brookfield viscosity of 50 to 2000 mPa-s, when measured at a 1 wt % concentration in water containing 0.53 wt % sodium carbonate at 20° C.

### DETAILED DESCRIPTION OF EMBODIMENTS OF THE INVENTION

Alginate is a polysaccharide and a structural component of brown seaweed. The building blocks of alginate are α-L-guluronic acid (G) and β-D-mannuronic acid (M). The M/G ratio depends on seaweed species and the part of the plant from which the alginate has been extracted. Suitable seaweeds include but are not limited to Laminaria hyperborea, Lessonia trabeculata, Lessonia nigrescens, Laminaria digitata, Macrocystis pyrifera, Ascophyllum nodosum, Laminaria japonica, antarctica, Durvillea potatorum, Eckonia maxima, as well as mixtures thereof. G blocks in the alginate can bind divalent cations such as Ca²⁺ to form an ionic crosslink between polymer chains. High-G alginates bind calcium ions stronger compared to high-M alginates. Commercially, alginates are extracted in an aqueous process and precipitated as alginic acid (E400). The alginic acid is neutralized with carbonate salts to form alginate salts such as sodium alginate (E 401), potassium alginate (E 402), ammonium (E 403) or calcium alginate (E 404). Calcium alginate is the only one of these four salts that is not soluble in water and requires interaction with a second component to bind or displace the calcium for the insoluble alginate to become functional in terms of creating any form of texture.

Pectic substances include pectins and pectates. Pectin is a naturally occurring polysaccharide found in the roots, stems, leaves, and fruits of various plants, especially the peel of citrus fruits such as limes, lemons, grapefruits, and oranges. Pectins contain polymeric units derived from D-galacturonic acid. About 20-60% of the units derived from D-galacturonic acid, depending on the source of the pectin, are esterified with methyl groups. These are commercially known as high methoxy pectin and low methoxy pectin, the latter also including amidated pectins. Pectate (pectinate) is fully de-esterified pectin with up to 20% of the units derived from D-galacturonic acid.

Carrageenan refers to a group of sulfated galactans extracted from red seaweed. Carrageenans are linear chains of D-galactopyranosyl units joined with alternating (1→3) α-D and (1→4) β-D-glycosidic linkages. Carrageenans may, in part, be distinguished by the degree and position of sulfation. Most sugar units have one or two sulfate groups esterified to a hydroxyl group at carbons C-2 or C-6. There are three main types of carrageenan, kappa carrageenan, iota carrageenan, and lambda carrageenan. Kappa carrageenans produce strong rigid gels with potassium ion while gels made with calcium ions and iota carrageenans are flaccid and compliant. Kappa-2-carrageenans form relatively weak gels with either calcium ions or potassium ions. Lambda carrageenans do not gel in water. A preferred carrageenan is iota carrageenan which is gelled with calcium ion. Iota carrageenan has a repeating unit of D-galactose-4-sulfate-3,6-anhydro-D-galactose-2-sulfate providing a sulfate ester content of about 25 to 34%.

The present invention is directed to insoluble polysaccharide salts which are gastro-activated dietary fibers. Insoluble polysaccharide salts are salts that have little or no negative impact during processing of an ingestible product containing such salts, and/or which exhibit good palatability in an ingestible product. The fiber properties of insoluble polysaccharide salts, such as the viscosity and water binding capacity, are primarily activated by the transit into the small intestine. The term activation here refers to going from an insoluble state to a viscous swollen or dissolved state which provides the functionality of a dietary fiber. One preferred insoluble polysaccharide salt is calcium alginate which is discussed in more detail below.

The insoluble polysaccharide salts may be added to ingestible compositions in an amount of from about 0.1 wt % to about 40 wt %. Amounts at the lower end of the range would potentially be used when the insoluble polysaccharide salts of the present invention are combined with one or more other viscosity-developing agents in the ingestible composition. Amounts at the higher end of the range may be used, for example, in supplements to provide a relatively large amount of fiber in a relatively small amount of ingestible composition.

The insoluble polysaccharide salts may also be used in amounts of from about 0.5 wt % to about 15 wt % of the ingestible, composition, from about 1.0 wt % to about 10 wt % of the ingestible composition, or from about 3 wt % to about 5 wt % of the ingestible composition. Calcium alginate powder may, for example, be used to provide the desired amount of insoluble polysaccharide salt.

The amount of the at least one gastro-activated dietary fiber included can vary, and will depend on the type of ingestible composition and the type of dietary fiber used. For example, typically a solid ingestible composition will include from about 0.25 g to about 10 g total dietary fiber per serving or any value therebetween. Another range of fiber intake in the compositions of this invention is about 0.5 g to about 8 g per serving, or about 1.0 to about 6 g per serving, or about 2.0 to about 5.0 g per serving. In certain cases, an ingestible product can include a soluble anionic fiber at a total amount from about 22% to about 40% by weight of the extruded product or any value therebetween.

A gastro-activated dietary fiber in accordance with the invention may include an insoluble polysaccharide salt selected from insoluble alginate salts, insoluble carrageenan salts, insoluble pectin salts and mixtures thereof. The polysaccharide salt should exhibit no swelling in water or a low degree of swelling in water. For example, the insoluble polysaccharide salt may have a water swelling ratio (V/V₀) of between 1 and 15, as measured after 24 hours in deionized water. In other embodiments, the water swelling ratio is between 1 and 10, or between 1 and 5. The swelling ratio (V/V₀) is the powder volume (as sediment) after 24 hours divided by the initial powder volume, as measured in a measuring cylinder charged with 2 grams of powder and 50 ml of water.

The gastro-activation of the dietary fiber to develop a viscosity increase may be induced by exposure to an acidic pH of 3 or less for at least 15 minutes and solubilizing ions. The polysaccharide salt is typically suitable for providing an increase in viscosity at a neutral pH within the range of 5.0 to 8.0, or a range of 6.5 to 7.6, after exposure to an acidic pH of 3 or less for at least 15 minutes. For example, in the case of a gastro-activated dietary fiber comprising a calcium alginate, the composition may show an increase in viscosity after 1 hour at pH in the range 6.9 to 7.6, after exposure to an acidic pH of 2.0 for 2 hours, for example, in the stomach.

It is also possible for the polysaccharide salt to provide an increase in viscosity without first being exposed to an acidic pH of 3 or less. In such a case, better viscosity development is provided at a pH above 7.0, or typically from 7.0-7.6. In each case, a monovalent ion, typically sodium, exchanges with the calcium or other insolubilizing ions for the polysaccharide salt to facilitate the viscosity development by solubilizing the polysaccharide salt. A suitable source of monovalent ion is provided naturally in the small intestine which excretes sodium bicarbonate.

Additional insolubilizing cations may be provided in the gastro-activated dietary fiber by addition of other salts containing the insolubilizing cations in order to elevate the cation content to higher concentrations which may reduce swelling and/or prevent premature activation of the gastro-activated dietary fiber.

The average molecular weight of the polysaccharide salt may range from 50,000-900,000 daltons; alternatively, from 100,000-600,000 daltons; or from 250,000-500,000 daltons. An average molecular weight of the polysaccharide salt of 350,000-450,000 daltons is particularly preferred. These molecular weight ranges apply to mixtures of two or more polysaccharide salts as well.

Calcium alginate powder is generally insoluble in water due to the interaction between the calcium and the G-blocks in the alginate chains. The individual particles may swell and absorb water to some extent depending on following three factors: (1) the M-block/G-block ratio of the alginate since high-G-block alginates will swell less than high M-block alginates; (2) the ratio of calcium to alginate; and (3) the presence of components that remove calcium ions from the G-blocks in the alginate since removal of calcium ions from the G-blocks in the alginate will increase swelling of the alginate. Preferably, the calcium alginate powder does not contain a substantial proportion of calcium alginate fibers and, more preferably, the calcium alginate powder is free of calcium alginate fibers. In one embodiment, the density of the gastro-activating calcium alginate dietary fiber is equal to or greater than 0.4 g/ml, as measured in a wide 10 ml measuring cylinder using a 10 ml sample with no powder compression.

The relative content of G and M monomers in the alginate polymers affects pore size, stability and biodegradability, gel strength and elasticity of gels. Alginate polymers may exhibit large variations in the total content of M and G, and the relative content of sequence structures also varies largely (G-blocks, M-blocks and MG alternating sequences) as well as the length of the sequences along the polymer chain. Gels with high G content alginate generally have stronger gel strengths relative to gels with high M-content alginate, which have lower gel strengths. The G content of the alginates may range from 25-75%, or from 35-70%. In general, higher G-block contents will tend to reduce the swelling of the gastro-activated dietary fiber, whereas lower G-block contents may make it easier to activate the gastro-activated dietary fiber. A balance of these properties may be desired depending upon the particular ingestible product.

Procedures for producing uronic blocks from are disclosed in U.S. Pat. No. 6,121,441. G-block alginate polymers and their uses as modulators of alginate gel properties is set forth in U.S. Pat. No. 6,407,226. Some preferred embodiments include at least 25% G-blocks, 30% G-blocks, 35% G-blocks, 40% G-blocks, 45% G-blocks, 50% G-blocks, 55% G-blocks, 60% G-blocks, 65% G-blocks, 70% G-blocks, 75% G-blocks, 80% G-blocks or 85% G-blocks.

In certain embodiments, a mixture of different molecular weight alginates can be used. For example, one or more high molecular weight alginates can be used in combination with one or more medium or low molecular weight alginates. An alginate blend can also be a mixture of a medium and low molecular weight alginate. The average molecular weight of the alginate may range from 50,000-900,000 daltons; alternatively, from 100,000-600,000 daltons; or from 250,000-500,000 daltons. An average molecular weight of the calcium alginate of 350,000-450,000 daltons is particularly preferred. These molecular weight ranges apply to mixtures of two or more alginates as well.

The particle size of the insoluble alginate may affect the gastro-activation process and the final properties of the solution. The smaller the particle size the more rapid the completion of gel formation. Larger particle sizes can influence the activation rate. Particle sizes may be controlled by, for example, by grinding and/or sifting insoluble alginate through various different sized filters such that the particles will generally be within a predetermined size range. In some embodiments, particles are up to about 125 μm, preferably up to about 75 μm for liquid products, and up to about (60 mesh upper limit for solid foods, more preferably, up to about 125 μm, and most preferably, up to about 75 μm. The lower limit of particle size is typically dictated by product mouthfeel and the desired viscosity of the food product.

When the polysaccharide salt is calcium alginate, the calcium alginate may have a Brookfield viscosity in the range of 50 to 2000 mPas when measured at 1 wt % calcium alginate in water containing 0.53% sodium carbonate at 20° C. Alternatively, the measured Brookfield viscosity of the calcium alginate may be in the range of 50 to 1000, in the range of 100 to 900 mPas, in the range of 200-600 mPas or in the range of 250-500 mPas.

A pectin can be a high-methoxy pectin (e.g., having greater than 50% esterified carboxylates), such as ISP HM70LV and CP Kelco USPL200. A pectin can exhibit any number average molecular weight range, including a low molecular weight range (about 100,000 to about 120,000 daltons, e.g., CP Kelco USPL200), medium molecular weight range (about 125,000 to about 145,000, e.g., ISP HM70LV), or high molecular weight range (about 150,000 to about 180,000, e.g., TIC HM Pectin). In certain cases, a high-methoxy pectin can be obtained from pulp, e.g., as a by-product of orange juice processing.

Alginates with lower calcium levels will typically swell more in water, and alginates with higher calcium levels will typically swell less in water. Also, calcium alginates will swell to a lesser extent in ingestible products that have soluble calcium salts available such as milk. Calcium alginates such as Protanal® TXF 200 (FMC BioPolymer) contain about 7 wt % Ca²⁺, corresponding to about an 85% saturation of the alginate monomers on a molar basis.

Sequestrants, chelating agents, monovalent salts, and/or sodium alginate may remove calcium ions from G-blocks in alginates and result in increased swelling in the presence of water, under certain conditions. High level of such components may eventually cause dissolution of the calcium alginate.

Swelling of the alginate in an ingestible product will affect the texture and palatability of the product. The aim of this invention is to add the right type of calcium alginate as a gastro-activated dietary fiber component in suitable types of ingestible products at the right conditions. This will give minimal swelling and no dissolution of the alginate, resulting in minimal or no changes in the texture and/or palatability of the ingestible product to which the fiber is added.

When ingesting an ingestible product, such as a food or beverage containing calcium alginate as a gastro-activated dietary fiber, most or all of the calcium alginate will remain insoluble in the mouth of the consumer since the pH is close to neutral and the residence time is short. Entering the stomach, the protons of the gastric acid will replace the calcium ions on the alginate chain, and the at least some of the alginate will be transformed from an insoluble calcium alginate to insoluble alginic acid or a mixture of insoluble calcium alginate and insoluble alginic acid. Upon stomach emptying into the small intestine, the stomach contents and the alginic acid are neutralized by sodium bicarbonate naturally secreted into the small intestine. The reaction of alginic acid and sodium bicarbonate yields soluble sodium alginate in the small intestine that will act as a soluble dietary fiber.

Thus, in one embodiment, powdered calcium alginate may be added to ingestible products with low or no salt content and no sequestering agents. In this embodiment, the calcium alginate would not interfere with the processing of the ingestible product and the palatability of the ingestible product will not be adversely affected to a noticeable degree.

It is also within the scope of the present invention to add the insoluble polysaccharide component to acidic ingestible products. Acidic ingestible products, such as foods or beverages, can potentially interact with calcium alginate during the processing of the ingestible product. At pH values lower than the pKₐ of alginates of 3.5, the calcium alginate will be partly or fully converted to alginic acid. As a result of this conversion, the alginate goes from one insoluble form to another, and thus the alginate particles will remain solid/insoluble under these acidic conditions. Data shows that swelling is affected very little by changes in acidic pH.

In some embodiments, it is desirable to add the insoluble polysaccharide component to salty ingestible products. Salty ingestible products containing salts such as sodium chloride may engage in ion exchange with the calcium in the alginate and alter the equilibrium concentrations to activate (solubilize) the alginate fiber. Using a higher G-block content materials and/or adding an extra amount of a calcium salt may counteract the monovalent ion/Ca²⁺ ion exchange reaction in the presence of salty ingestible products and reduce or prevent activation of the polysaccharide component in these types of products.

Similarly, in ingestible compositions containing water, using a higher G-block content materials and/or adding an extra amount of a calcium salt may counteract the monovalent ion/Ca²⁺ ion exchange reaction in the presence of water and reduce or prevent activation of the polysaccharide component in these types of products.

Sequestrants such as phosphates, EDTA and/or citrates in ingestible products may chelate the calcium ions of calcium alginate and activate (solubilize) the alginate fiber. Again, using an alginate with high G-block content and/or adding an extra amount of a calcium salt may counteract the Ca²⁺ ion chelation and reduce or prevent activation. Sequestrants are normally added to the products in order to bind divalent ions, and their abundance may be needed to balance the formulation. Thus, in some embodiments, small amounts of sequestrants may be present in compositions employing the present invention.

Alternatively, the polysaccharide salt component may be provided with a suitable coating to substantially reduce or prevent contact of water with the polysaccharide salt. Such a coating would reduce or prevent ion exchange between monovalent salts and the polysaccharide salt and/or reduce or prevent chelation by sequestrants. Suitable coatings should dissolve when exposed to the contents of the mouth, stomach and/or small intestine so as to permit activation of the polysaccharide salt in the small intestine. Conventional wax-based coatings are an example of a type of coating which may be suitable for this purpose.

Many factors play a role when selecting the alginate raw material. If it is desired to use an alginate with moderate or even low G-block contents, for instance those that can be isolated from Laminaria hyperborea leaves or Lessonia nigrescens, respectively, there is a higher risk of activating the calcium alginate fiber during processing or in the mouth of the consumer. Ultrapure alginate may also be employed in some embodiments.

To reduce the risk of activation, a higher calcium level in the product can be used or an extra amount of a calcium salt can be provided in the composition and/or ingestible product. The gastro-activated dietary fiber may contain from 5 to 12 wt % cation, based on the total weight of the polysaccharide salt. Alternatively, the gastro-activated dietary fiber may contain 6-10 wt % cation, or 7-9 wt % cation, based on the total weight of the polysaccharide salt. Thus, for example, these amounts apply to the amount of calcium present in calcium alginate salts used in the gastro-activated dietary fiber compositions of the present invention. Alternatively, additional calcium salts may be added to the gastro-activated dietary fiber to increase the calcium content. All references to the cation content refer to cations which are suitable for rendering the polysaccharide salt insoluble or substantially insoluble in water and which provide the desired swelling ratios discussed above. In some embodiments, higher contents of cation are desired, potentially for one or more of the reasons discussed above. In such embodiments, the cation, for example, calcium with calcium alginate, may comprise, for example, 9-12 wt % of the polysaccharide salt.

In some embodiments, it is preferred that methods of manufacture of insoluble gastro-activated dietary fiber compositions provide polysaccharide salts with a stoichiometric amount (100% saturation) of cations. In some embodiments, the method of manufacture of insoluble gastro-activated dietary fiber compositions may provide polysaccharide salts with sub-stoichiometric amount (<100% saturation) of said cation.

Commercial calcium salts of alginic acid are generally manufactured in processes whereby calcium is added to alginic acid in the solid phase by simple admixture and kneading of the components together. Examples of commercially available calcium salts of alginic acid are Protaweld® (from FMC BioPolymer) and Kelset® from ISP Corporation.

Polysaccharide salts generally have a cation such as, for example, calcium or strontium, barium, zinc, iron, manganese, potassium, copper, cobalt, nickel, or combinations thereof. Insoluble alkaline earth salts of alginic acid such as for example calcium alginate or strontium alginate, or insoluble transition metal salts of alginic acid (such as those using cations of copper, nickel, zinc, iron, manganese or cobalt) can be manufactured with a known and predetermined content of alkaline earth ions by precipitation from the solutions. In some embodiments, sodium alginate is used to in the process of preparing an insoluble alginate salt.

A salt containing the desired cation for the insoluble alginate, such as for example, calcium salt or strontium salt such as calcium chloride, calcium lactate or strontium chloride, is used to make a solution. A small amount of a sodium alginate solution is combined, preferably slowly, with the cation solution. Preferably, the combined solutions are continuously stirred during the mixing process. Insoluble alginate such as for example calcium alginate or strontium alginate (depending upon the gelling ion used) precipitates from the combined solutions. The precipitated insoluble alginate is then be removed from the solution and washed repeatedly, such as 2-10 times, with purified water for example to remove all soluble ions.

The removal of soluble ions is confirmed for example by testing the conductivity of insoluble alginate in purified water compared to the conductivity of purified water. After washing, the insoluble alginate can be dried, such as with a vacuum. The dried alginate can be milled and, in some embodiments, selected for particle sizes. Another suitable manufacturing process for making insoluble alginate salts is disclosed, for example, in U.S. Pat. No. 1,814,981, the disclosure of which manufacturing process is hereby incorporated by reference.

Alternatively, rather than, or in addition to, adjusting the cation content of the polysaccharide salt, the cation content of the gastro-activated dietary fiber and/or the cation content of the ingestible product may be adjusted to customize the properties of the gastro-activated compositions or ingestible products containing them. Such adjustments can be made by adding additional salts of the desired cation. For example, calcium chloride can be added to the gastro-activated dietary fiber composition or the ingestible product to increase the calcium cation content thereof. Other suitable salts for this purpose include food grade salts that include an insolubilizing cation for the polysaccharide. Exemplary additional food grade salts such as calcium lactate and calcium acetate. The amount of additional salt employed depends primarily on the desired cation content of the final product. In this manner, greater than 100% of the stoichiometric amount of the cation required to form the polysaccharide salt can be provided to the dietary fiber or ingestible composition. This provides the advantage that further adjustments can be made to compensate for, for example, aqueous, acidic or salty ingestible compositions by providing an excess of cation which drives the ion exchange equilibrium in favour of maintaining the salt or insoluble form of the gastro-activated dietary fiber until activation in the small intestine is desired.

The gastro-activated dietary fiber of the present invention is particularly useful for ingestible products such as foods and beverages. One class of foods in which the gastro-activated dietary fiber are processed foods. A variety of different food products can be prepared including baked goods such as breads, bakery creams, candies, sweetened beverages, health drinks, flavoured waters, fried food products and soy beverages.

One or more gastro-activated dietary fibers can be present in a solid ingestible composition in either a processed or unprocessed form. Ingestible compositions containing gastro-activated dietary fibers can be processed, without limitation, by extrusion (cold or hot, high pressure or low pressure), spray-drying, roll-drying, dry-blending, roll-blending, freeze-drying, blending, mixing, high-shear mixing, baking, boiling, frying and fermentation processes, homogenization and ultra-high temperature (UHT) processing. One or more gastro-activated dietary fibers can be present in a solid ingestible composition in one or more processed forms (e.g., an extruded food product, such as a crispy as described below) or in an unprocessed forms (e.g., a formed dough or composition), or both. For example, a snack bar solid ingestible composition can include one or more gastro-activated dietary fibers present as an extruded food product, one or more gastro-activated dietary fibers in an un-extruded form (e.g., a formed bar), or both. A snack chip solid ingestible composition can include one or more gastro-activated dietary fibers in extruded form or in spray-dried form, or both, e.g., an extruded gastro-activated dietary fiber-containing chip having one or more gastro-activated dietary fibers spray-dried on the chip. A cookie solid ingestible composition can include one or more gastro-activated dietary fibers in an unprocessed form (e.g., a formed cookie) or in a processed (e.g., extruded) form, or both.

A solid ingestible composition can include optional ingredients such as frostings, coatings, drizzles, chips, chunks, swirls, or layers. Such optional ingredients can be a source of one or more cations, as described further herein. Such optional ingredients can also be a source of one or more gastro-activated dietary fibers, e.g., pectin in a jelly layer.

A solid ingestible composition can include an extruded food product. An extruded food product can be cold- or hot-extruded under high or low pressure and can assume any type of extruded shape, including without limitation, a bar (e.g., a nutritional bar or meal replacement bar), cookie, bagel, crispy, puff, curl, crunch, ball, flake, square, nugget, and chip. In some cases, an extruded food product is in bar shape, such as a snack bar, granola, nutritional bar, or meal replacement bar. In some cases, an extruded food product is in cookie shape. In other cases, an extruded food product is in a shape such as a crispy, puff, flake, curl, ball, crunch, nugget, chip, square, chip, pasta, or nugget. Such extruded food products can be eaten as is (e.g., cookies, bars, chips, crispies as cereal) or can be incorporated into a solid ingestible composition, e.g., crispies incorporated into snack bars.

An ingestible composition or extruded food product can include one or more of the following: cocoa, other insoluble or soluble fibers, insoluble cellulosic material (e.g., microcrystalline cellulose, such as Avicel® (FMC, Philadelphia, Pa.) or Solka Floc® (International Fiber Corporation, North Tonawanda, N.Y.)), and oils or fats derived from animal or vegetable sources, e.g., soybean oil, canola oil, corn oil, safflower oil, sunflower oil, palm, palm kernel, etc. For example, an extruded food product can include about 3% to about 10% (e.g., about 3% to about 6%, about 4% to about 6%, about 5%, about 6%, about 7%, or about 4% to about 8%) by weight of such an added ingredient.

The present invention also encompasses liquid ingestible compositions. A liquid ingestible composition may have any suitable pH. However, liquid ingestible compositions preferably have a pH from about 2.5 to about 7.5. In certain cases, a liquid ingestible composition can have a pH from about 3.0 to about 7, e.g., about 4.0 to about 4.3, or about 4.1 to about 4.2. At these pHs, it is believed that the liquid ingestible compositions are above the pKas of the alginate and pectin acidic subunits, minimizing ion exchange in the composition. In some cases, malic, phosphoric, and citric acids can be used to acidify the compositions. In certain cases, a liquid ingestible composition may have a pH of from about 4.0 to about 6.5. Such liquid ingestible compositions can use pH buffers known to those having ordinary skill in the art.

Sweeteners for use in a liquid ingestible composition can vary according to the use of the composition. For diet beverages, low glycemic sweeteners and/or high intensity sweeteners may be preferred, such as polyols, trehalose, isomaltulose, and sucralose. Sucralose and/or other high intensity sweeteners such as aspartame, neotame, acesulfame K, etc. can be used alone in certain formulations. The choice of sweetener will impact the overall caloric content of a liquid ingestible composition. In certain cases, a liquid ingestible composition can be targeted to have about 40 calories/12 oz serving.

A liquid ingestible composition can exhibit a viscosity in the range of from about 15 to about 200 cPs, or any value therebetween, at a shear rate of about 10⁻⁵, e.g., about 17 to about 24; about 20 to about 25, about 50 to 100, about 25 to 75, about 20 to 80, about 15 to about 20, about 100 to about 200, about 125 to about 175, about 150 to about 175 cPs. Viscosity can be measured by one having ordinary skill in the art, e.g., by measuring flow curves of solutions with increasing shear rate using a double gap concentric cylinder fixture (e.g., with a Parr Physica Rheometer).

A liquid ingestible composition can include a cation sequestrant, selected from EDTA and its salts, sodium citrate, sodium hexametaphosphate, sodium acid pyrophosphate, trisodium phosphate anhydrous, tetrasodium pyrophosphate, sodium tripolyphosphate, disodium phosphate, sodium carbonate, and potassium citrate, so long as the amount of cation sequestrant does not chelate an amount of cations from the polysaccharide salt which will cause substantial solubilization of the polysaccharide salt in the liquid composition. A cation sequestrant can be from about 0.0001% to about 0.3% of the ingestible composition. Thus, for example, EDTA can be used at about 0.00015 to about 0.1% by weight.

A liquid ingestible composition can include a juice or juice concentrate and optional flavorants and/or colorants. Juices for use include fruit juices such as apple, grape, raspberry, blueberry, cherry, pear, orange, melon, plum, lemon, lime, kiwi, passion fruit, blackberry, peach, mango, guava, pineapple, grapefruit, and others known to those having ordinary skill in the art. Vegetable juices for use include tomato, spinach, wheatgrass, cucumber, carrot, peppers, beet, aloe and others known to those of ordinary skill in the art.

Flavorants can be included depending on the desired final flavor, and can include flavors such as kiwi, passion fruit, pineapple, coconut, lime, creamy shake, peach, pink grapefruit, peach grapefruit, pina colada, grape, banana, chocolate, vanilla, cinnamon, apple, orange, lemon, cherry, berry, blueberry, blackberry, apple, strawberry, raspberry, melon(s), coffee, and others, available from David Michael, Givaudan, Duckworth, and other sources. Colorants can also be included depending on the final color to be achieved.

In ingestible products, calcium alginate can be added to the formulation, by for instance dry mixing the powder with other dry ingredients. Because of the low swelling and absorption of water, the risk of lumping or aggregation of calcium alginate is much less compared to using hydrocolloids that dissolve in water. This would facilitate processing of ingestible products using this ingredient. In a beverage, the calcium alginate powder will suspend easily. However, under some circumstances, the powder may form sediment. In such a case, sedimentation can be prevented or reduced by use of a suitable suspending agent such as colloidal microcrystalline cellulose or a non-ionic soluble hydrocolloid such as xanthan.

In acidic ingestible products the molecular weight of the alginate will decrease upon storage. This will not change the palatability of the ingestible product since calcium alginates with lower molecular weights swell less than calcium alginates with higher molecular weights. However, the reduction of molecular weight after a significant storage period in the presence of an acid environment may slightly reduce the viscosity enhancing effect of the gastro-activated dietary fiber.

### EXAMPLES

### Example 1

Calcium alginates having a range of calcium contents and alginate types were prepared by kneading alginic acid derived from various seaweeds with an appropriate blend of calcium carbonate and sodium carbonate. TP1, TP2, TP3, TP4, TP5, TP6, TP7 and TP8 were manufactured using a base lot of alginic acid with a 95/5 mixture of calcium carbonate and sodium carbonate plus an additional 10%, 20%, or 40% of calcium added as calcium chloride. Additional water was added during manufacture of TP5 and TP6 to ensure uniform mixing.

The alginates were characterized for swelling in water as a function of time and for Brookfield viscosity in 0.53% sodium carbonate in water (to bind calcium as calcium carbonate). Swelling was rapid. Data are reported after 24 hours to allow measurement of a sedimentation volume. Several samples with low calcium content had a high swellability. The TP samples all showed low swellability.

TABLE 1
Calcium Alginate

| | | | Calcium | | | Swelled in |
|---|---|---|---|---|---|---|
| | Calcium | | Content | | | water 24 |
| Sample | Alginate | Raw material | wt %¹⁾ | Viscosity²⁾ | | hrs (V/V₀)³⁾ |
| 1 | RENO 1001 | L. hyperborean, stem | 6.28% | 88 | mPas | 3.7 |
| 2 | RENO 3056 | L. hyperborea, stem | 6.37% | 269 | mPas | 10.5 |
| 3 | RENO 3051 | L. hyperborea, leaf | 5.50% | 255 | mPas | Swells |
| | | | | | | much, does |
| | | | | | | not |
| | | | | | | sediment |
| 4 | RENO 3052 | Lessonia trabeculata | 5.52% | 134 | mPas | Swells |
| | | | | | | much, does |
| | | | | | | not |
| | | | | | | sediment |
| 5 | 530021 | Lessonia trabeculata | 5.00% | 160 | mPas | 48 |
| 6 | 530022 | Lessonia trabeculata | 6.12% | 171 | mPas | 45 |
| 7 | 630017 | L. hyperborea, stem | 6.02% | 245 | mPas | 12 |
| 8 | 630030 | L. hyperborea, stem | 6.31% | 300 | mPas | 19 |
| 9 | 630034 | L. hyperborea, stem | 6.82% | 342 | mPas | 13 |
| 10 | TP 1 | L.hyperborea, leaf | 10.4%⁴⁾ | 566 | mPas | 2.0 |
| | | Lessonia nigrescens | | | | |
| 11 | TP 2 | L. hyperborea, leaf | 11.4%⁴⁾ | 382 | mPas | 1.8 |
| | | Lessonia nigrescens | | | | |
| 12 | TP 3 | L. hyperborea, stem | 10.4%⁴⁾ | 110 | mPas | 1.7 |
| 13 | TP 4 | L. hyperborea, stem | 11.4%⁴⁾ | 106 | mPas | 1.7 |
| 14 | TP 5 | L. hyperborea, stem | 9.8%⁴⁾ | 70 | mPas | 1.5 |
| 15 | TP 6 | L. hyperborea, stem | 10.4%⁴⁾ | 88 | mPas | 1.6 |
| 16 | TP 7 | L. hyperborea, stem | 11.4%⁴⁾ | 191 | mPas | 1.9 |
| 17 | 10614 | L. hyperborea, stem | Not tested | 272 | mPas | Not tested |
| 18 | TP8 | L. hyperborea, leaf | 8.4%⁴⁾ | 900 | mPas | 2.5 |

¹⁾Determined by using atomic absorption
²⁾Determined by dissolving 1% calcium alginate in water containing 0.53% sodium carbonate.
³⁾Powder volume (sediment) after 24 hours over initial powder volume, all measured in a measuring cylinder charged with 2 grams of powder and 50 ml of water.
⁴⁾Calculated based on ingredients added into the small-scale batch process.

### Example 2

This example shows a simulated “in vivo” activation of calcium alginate. 0.4 g of calcium alginate, RENO 3051, was added and stirred in 15 ml of 0.1 M HCl for 10 minutes to simulate a short stay at gastric conditions. The alginate remained insoluble. Sodium bicarbonate was added (0.4 g) until the pH was 7.5 to simulate the entry into the small intestine. It was seen that the particles swell and some viscosity was created. Over time the viscosity continued to develop significantly.

### Example 3

This example shows that the swelling of calcium alginate in water is fairly independent of acidic to neutral pH. Calcium alginates, isolated from Laminaria hyperborea stem were suspended in deionized water before adjusting pH to values ranging from acidic to neutral. The volume of the powder was noted on the dry powder (Vₒ), and several times during the swelling study. After 24 hours the swelling curve had stabilized for all samples. The data in Table 2 show variations in swelling ratio (V/Vₒ) over the pH range after 24 hours, but no large differences were observed.

TABLE 2
24 hour Swelling Ratio of calcium alginates as a function of pH

| pH | RENO 1001 | RENO 3056 |
|---|---|---|
| Not adjusted | 3.7 | 10.5 |
| 5 | 4.3 | 10.3 |
| 4 | 6.6 | 9.6 |
| 3 | 4.7 | 11.9 |

### Example 4

Food processing often requires high temperatures and/or high shear. This example shows the swelling of calcium alginates after various process treatments. After suspending 2 grams of the calcium alginate (RENO 1001) in 50 grams of water the samples were brought to boiling using a microwave oven. Two of the samples were then additionally mixed with a high shear Silverson® (mixer equipped with a rotor/stator. The high shear mixing may have attrited or deaggregated the particles, reducing the particle size since the sedimentation of the particles took a longer time. Table 3 shows the swelling ratio (V/Vₒ) of the powder after 48 hrs. This shows that the swelling of alginates is impacted to a small extent by heat treatment alone (boiling). High shear increases the swelling, but the results may have also been impacted by a change in particle size and structure, again impacting the sedimentation process.

TABLE 3
Swelling of calcium alginate as a function of process treatment

| Treatment | Swelling (V/Vₒ) |
|---|---|
| No treatment (cold water) | 3.7 |
| Boiled | 5.6 |
| Boiled and high shear mixing for 1 minute | 9.4 |
| Boiled and high shear mixing for 20 minutes | 15.1 |

### Example 5

This example shows incorporation of calcium alginate into bread. Calcium alginate (batch 612321) based on a high G-block content from Laminaria hyperborea stem was used in this trial. The reference bread was made by mixing and kneading 1000 g of wheat flour, 50 g dry yeast, 30 g sugar, 4 tablespoons of soybean oil and 1.5 teaspoons of NaCl in 700 ml tempered water. For the alginate containing bread the amount of wheat flour was reduced to 797 grams and 70 grams of calcium alginate was added. Both the samples of dough were raised, then kneaded a second time before being added to the moulds. The consistency of the bread dough with alginate was somewhat less sticky than the dough of the reference bread. After 30 minutes on the bench, the two breads were baked at 225° C. for approximately 40 minutes. The two breads looked almost the same and tasted very similar. The alginate bread kept the shape better during the baking process. The bread volume of the reference bread was somewhat higher than the alginate bread. The core of the reference bread was slightly stickier compared to the alginate bread. During baking, the weight of the alginate bread was reduced from 1599 to 1486 grams whereas the weight of the reference bread was reduced from 1692 to 1587 grams. The dry matter was determined in an infrared drier at 140° C. for 20 minutes to be 56.2% and 58.5% for the alginate and reference bread, respectively.

### Example 6

This example demonstrates gastric activation of calcium alginate in a food product. Samples (about 50 g to 75 g) of the bread made in Example 4 were added to an amount of 0.1 M HCl (pH=1) corresponding to twice the bread sample weight and mixed in a food processor for 5 minutes to simulate the gastric conditions. The viscosity was measured on the blend to be 2360 mPas for the alginate bread and 655 mPas for the reference bread, both measured at 23° C. The pH was adjusted to within the range 6.5 to 7 with sodium bicarbonate. Then the samples were heated to 37° C., and sodium carbonate was added to neutral pH to simulate the entry into the small intestine. The viscosity was now measured was 2795 mPas for the alginate bread and 540 mPas for the reference bread.

### Example 7

This example shows calcium alginate dietary fiber incorporated into white bread with and without added calcium. Bread was prepared according to the recipe in Table 4. All the dry ingredients were mixed together and added to lukewarm water with soybean oil. For Bread 3 the calcium lactate was dissolved in the water. The dough was mixed using a Kenwood Major® mixer and then kneaded by hand. Extra water was added to breads 2 and 3 in the mixing step to provide approximately the same texture of the dough. The dough was raised for 30 minutes at the bench. Then the dough was cut in two and placed in to the two baking tins. “After raising” in 20 minutes at the bench, then the bread was baked at 225° C. for approximately 45 minutes.

TABLE 4
Bread formulations

| | Bread 1 | | |
|---|---|---|---|
| | (control) | Bread 2 | Bread 3 |
| Recipe | | | |
| Water [g] | 700 | 940 | 800 |
| Ca-alginate (RENO 3056) [g] | 0 | 134.7 | 134.7 |
| Ca-lactate [g] | 0 | 0 | 7.76 |
| Soybean oil [g] | 27 | 27 | 27 |
| Dry yeast [g] | 50 | 50 | 50 |
| Sugar [g] | 30 | 30 | 30 |
| NaCl [g] | 9 | 9 | 9 |
| Wheat flour [g] | 1000 | 1000 | 1000 |
| Results | | | |
| Weight of bread dough before baking, [g] | 1774 | 2151 | 2021 |
| Weight of bread after baking, [g] | 1627 | 2033 | 1859 |
| Weight reduction during baking, [%] | −8.3 | −5.5 | −8.0 |
| Density, [g/cm3] | 0.39 | 0.49 | 0.52 |
| Dry matter*, [%] | 61.6 | 53.1 | 58.8 |
| Content of alginate, [%] | 0 | 6.2 | 6.6 |

*Loss in drying using an infrared drier, SME-2081, drying temperature 140° C. in 20 minutes.

The taste and the appearance were almost the same for all three breads. Bread 1 was slightly more coherent than the two other breads. The textures of the breads were similar to each other. The density measurements show that the reference bread rose the most of the three breads. The bread dough with calcium alginate needed more water than either the reference bread or the bread with calcium alginate and calcium lactate. This corresponds to the indication of the amount of dry matter given in the table.

### Example 8

This example demonstrates activation of the calcium alginate in the bread by measurement of viscosity at simulated gastro-intestinal conditions. 50 g of bread made in accordance with Example 7 was added to 150 g 0.1 M HCl (pH 1) in a ratio of 1:3 and mixed five minutes in a food processor to simulate gastric pH conditions. The viscosity of the mixture was measured using a Brookfield RV viscometer right away, after one hour and after two hours. Then the pH was adjusted close to pH 7 with sodium bicarbonate to simulate intestinal pH conditions. The viscosity was then measured right away, after one hour and after two hours.

TABLE 5
Viscosity under Simulated Gastrointestinal Conditions

| | Bread No. | | | | | |
|---|---|---|---|---|---|---|
| | Bread 1 | | Bread 2 | | Bread 3 | |
| | Comment | | | | | |
| | The | | Bread with | | Bread calcium | |
| | reference | | calcium | | alginate and | |
| | bread | | alginate | | calcium lactate | |
| | 1A | 1B | 2A | 2B | 3A | 3B |
| Sample weight, | 50.2 | 50.4 | 50.1 | 50.2 | 50.2 | 50.3 |
| [g] | | | | | | |
| Weight of | 150.7 | 151.2 | 150.3 | 150.6 | 150.6 | 150.9 |
| 0.1M H [g] | | | | | | |
| Gastric test | Simulated gastric viscosity, [mPa*s], | | | | | |
| | Temperature: 21-22° C. | | | | | |
| Right away | 67 | 45 | 65 | 60 | 66 | 55 |
| After 1 hour | 85 | 50 | 65 | 65 | 80 | 65 |
| After 2 hours | 100 | 60 | 75 | 70 | 85 | 70 |
| Adjust pH | | | | | | |
| Starting pH | 1.30 | 1.40 | 1.92 | 2.03 | 2.11 | 2.20 |
| Ending pH | 6.91 | 6.97 | 6.92 | 6.92 | 6.92 | 6.98 |
| Intestinal test | Simulated intestinal viscosity, [mPa*s] | | | | | |
| | Temperature: 37° C. | | | | | |
| Right away | 135 | 155 | 165 | 160 | 285 | 155 |
| After 1 hour | 190 | 170 | 325 | 330 | 510 | 287 |
| After 2 hours | 290 | 225 | 442 | 430 | 850 | 645 |

### Example 9

This example shows simulated gastro-activation of calcium alginate. The simulated conditions were determined by monitoring viscosity and pH with time. Calcium alginate was added and stirred in 0.1 M HCl for 2 hours to simulate gastric conditions. The alginate remained insoluble. Sodium bicarbonate was added until the pH was 7.0 to simulate the entry into the small intestine. After one hour, additional sodium bicarbonate was added to raise the pH. Not all samples were adjusted to pH 7.5. Following the study, the samples were refrigerated overnight and observed the following morning. Data are reported in Tables 6, 7 and 8.

Two samples were not gastro-activated under these test conditions: 530022 and 630030. Two samples were mildly gastro-activated: 630034 and RENO 1001. Four samples were gastro-activated and had an increased viscosity under the test conditions: 530021, RENO 3051, TP1 and TP6.

TABLE 6
Gastro-activation Testing of Calcium Alginates

| | Sample | | | | | |
|---|---|---|---|---|---|---|
| | 530021 | | 530022 | | 630030 | |
| | Water swell | | | | | |
| | 48 | | 45 | | 19 | |
| Property | viscosity | pH | viscosity | pH | viscosity | pH |
| Gastric test | Simulated gastric viscosity, [mPa*s], | | | | | |
| | Temperature: 30-35° C. | | | | | |
| Right away | 2.0 | 1.79 | 2.0 | 1.78 | 2.0 | 1.91 |
| After 1 hr | 2.0 | 1.79 | 2.0 | 1.8 | 2.0 | 1.92 |
| After 2 hrs | 2.0 | 1.79 | 2.0 | 1.82 | 2.0 | 1.94 |
| Intestinal test | Adjust pH to 7 | | | | | |
| | Simulated intestinal viscosity, [mPa*s] | | | | | |
| | Temperature: 37° C. | | | | | |
| Right away | 2.0 | 6.95 | 2.0 | 6.92 | 2.0 | 6.97 |
| After 1 hr | 2.0 | 7.09 | 2.0 | 7.14 | 6.0 | 7.09 |
| Intestinal test | Adjust to 7.5 | | | | | |
| | Simulated intestinal viscosity, [mPa*s] | | | | | |
| | Temperature: 37° C. | | | | | |
| Right away | 8.0 | 7.54 | 2.0 | 7.58 | 4.0 | 7.54 |
| After 1 hour | 480 | 7.2 | 2.0 | 7.47 | 2.0 | 7.33 |
| Total added | 7.87 g | | 6.79 g | | 7.78 g | |
| NaHCO3 | | | | | | |
| Overnight | dissolved | | 40% ppt | | 80% ppt | |

TABLE 7
Gastro-activation of Calcium Alginate samples

| | Sample | | | | | |
|---|---|---|---|---|---|---|
| | 60034 | | RENO 1001 | | RENO 3051 | |
| | Water swell | | | | | |
| | | | | | Swells, does | |
| | 13 | | 3.7 | | not sediment | |
| Property | viscosity | pH | viscosity | pH | viscosity | pH |
| Gastric test | Simulated gastric viscosity, [mPa*s], | | | | | |
| | Temperature: 30-35° C. | | | | | |
| Right away | 2.0 | 1.92 | 3.0 | 1.78 | 4.0 | 1.96 |
| After 1 hr | 2.0 | 1.96 | 2.0 | 1.8 | 2.0 | 2.01 |
| After 2 hrs | 2.0 | 1.99 | 2.0 | 1.82 | 2.0 | 2.03 |
| Intestinal test | Adjust pH to 7 | | | | | |
| | Simulated intestinal viscosity, [mPa*s] | | | | | |
| | Temperature: 30-35° C. | | | | | |
| Right away | 2.0 | 6.94 | 3.0 | 6.92 | 2.0 | 6.93 |
| After 1 hr | 2.0 | 7.03 | 2.0 | 7.14 | 6.0 | 7.05 |
| Intestinal test | Adjust to 7.5 | | | | | |
| | Simulated intestinal viscosity, [mPa*s] | | | | | |
| | Temperature: 30-35° C. | | | | | |
| Right away | 8.0 | 7.50 | 16.0 | 7.58 | 398 | 7.30 |
| After 1 hour | 30 | 7.12 | 35.0 | 7.47 | 5930 | 7.17 |
| Total added | 6.72 g | | 6.91 g | | 9.65 g | |
| NaHCO3 | | | | | | |
| Overnight | 80% ppt | | 80% ppt | | Viscous | |
| (refrigerated) | | | | | Partial gel | |

TABLE 8
Gastro-activation of Calcium Alginate Samples

| Sample | |
|---|---|
| TP1 | TP6 |
| Water swell | |
| 2.0 | 1.6 |

| Property | viscosity | pH | viscosity | pH |
|---|---|---|---|---|
| Gastric test | Simulated gastric viscosity, [mPa*s], | | | |
| | Temperature: 30-35° C. | | | |
| Right away | 4.0 | 2.02 | 8.0 | 2.01 |
| After 1 hr | 2.0 | 2.21 | 2.0 | 2.02 |
| After 2 hrs | 2.0 | 2.24 | 2.0 | 2.04 |
| Intestinal test | Adjust pH to 7 | | | |
| | Simulated intestinal viscosity, [mPa*s] | | | |
| | Temperature: 30-35° C. | | | |
| Right away | 3.0 | 6.94 | 26 | 6.98 |
| After 1 hr | 20.0 | 6.94 | 986 | 6.92 |
| Intestinal test | Adjust to 7.0 to 7.2 | | | |
| | Simulated intestinal viscosity, [mPa*s] | | | |
| | Temperature: 30-35° C. | | | |
| Right away | 322 | 7.16 | 1070 | 6.92 |
| After 1 hour | 3275 | 7.10 | 2346 | 6.92 |
| Total added | 10.41 g | | 7.71 g | |
| NaHCO3 | | | | |
| Overnight | Viscous, | | Viscous, | |
| (refrigerated) | some | | some | |
| | particles | | particles | |

### Example 10

This example is directed to a palatable beverage prepared with a gastro-activated calcium alginate. A soy beverage was prepared with and without 2.5% calcium alginate. A stabilizer containing microcrystalline cellulose and cellulose gum was included to suspend the insoluble calcium alginate. The dry ingredients were blended together and dispersed in the fluid soymilk base using a wire whisk. The mix was heated to 90° C. and kept at this temperature for 10 minutes. After cooling down to 75° C., both samples were homogenized at 130/20 bars using a PONY NS2006L Energy (GEA Niro Soavi) homogeniser. The product was further cooled down to 8° C., poured into bottles and stored under refrigeration. Alternatively, the beverage can be processed on an industrial UHT unit, with a heat treatment of 140° C. for 3 seconds and then filled aseptically into bottles to provide long shelf life beverages.

Having described embodiments of the invention which are intended to be illustrative and not limiting, it is noted that modifications and variations can be made by persons skilled in the art in light of the above teachings. It is therefore to be understood that changes may be made in the particular embodiments of the invention disclosed which are within the scope and spirit of the invention as outlined by the appended claims. Having thus described the invention with the details and particularity required by the patent laws, the intended scope of protection is set forth in the appended claims.

We claim:

1. A palatable gastro-activated dietary fiber comprising an insoluble polysaccharide salt selected from the group of insoluble alginate salts, insoluble carrageenan salts, insoluble pectin salts and mixtures thereof, said polysaccharide salt being low or non-swelling in water and increasing in viscosity at a neutral pH within the range 6.5 to 7.6 after exposure to an acidic pH of 3 or less for at least 15 minutes.

2. The palatable gastro-activated dietary fiber of claim 1, comprising an insoluble polysaccharide salt having a water swelling ratio of between 1 and 15 measured after 24 hours in deionized water.

3. The palatable gastro-activated dietary fiber of claim 1, comprising an insoluble polysaccharide salt having a water swelling ratio of between 1 and 10 measured after 24 hours in deionized water.

4. The palatable gastro-activated dietary fiber of claim 1, comprising an insoluble polysaccharide salt having a water swelling ratio of between 1 and 5 measured after 24 hours in deionized water.

5. The palatable gastro-activated dietary fiber of claim 1, comprising 5-12 weight percent of a cation which renders the polysaccharide insoluble, based on the total weight of the gastro-activated dietary fiber.

6. The palatable gastro-activated dietary fiber of claim 1, comprising 6-10 weight percent of a cation which renders the polysaccharide insoluble, based on the total weight of the gastro-activated dietary fiber.

7. The palatable gastro-activated dietary fiber of claim 1, comprising 7-9 weight percent of a cation which renders the polysaccharide insoluble, based on the total weight of the gastro-activated dietary fiber.

8. The palatable gastro-activated dietary fiber of claim 1, wherein the polysaccharide salt has a molecular weight of from about 50,000 to about 900,000 daltons.

9. The palatable gastro-activated dietary fiber of claim 1, wherein the polysaccharide salt has a molecular weight of from about 100,000 to about 600,000 daltons.

10. The palatable gastro-activated dietary fiber of claim 1, wherein the polysaccharide salt has a molecular weight of from about 250,000 to about 500,000 daltons.

11. The palatable gastro-activated dietary fiber of claim 1, wherein the polysaccharide salt has a molecular weight of from about 350,000 to about 450,000 daltons.

12. The palatable gastro-activated dietary fiber of claim 1, further comprising an additional salt of a cation that renders the gastro-activated dietary fiber insoluble.

13. The palatable gastro-activated dietary fiber of claim 1, comprising an insoluble alginate salt derived from the stem or leaves of seaweeds selected from the group Laminaria hyperborea, Lessonia trabeculata, Lessonia nigrescens, Laminaria digitata, Macrocystis pyrifera, Ascophyllum nodosum, Laminaria japonica, Durvillea antarctica, Durvilliea potatorum, Eckonia maxima, and mixtures thereof.

14. The palatable gastro-activated dietary fiber of claim 2, wherein the insoluble polysaccharide salt comprises calcium alginate.

15. The palatable gastro-activated dietary fiber of claim 14, further comprising an additional calcium salt.

16. The palatable gastro-activated dietary fiber of claim 15, wherein the additional calcium salt is selected from the group consisting of calcium chloride, calcium lactate and calcium acetate.

17. The palatable gastro-activated dietary fiber of claim 14, wherein the alginate comprises at least 25% by weight of G-blocks.

18. The palatable gastro-activated dietary fiber of claim 14, wherein the alginate comprises at least 40% by weight of G-blocks.

19. The palatable gastro-activated dietary fiber of claim 14, wherein the alginate comprises at least 60% by weight of G-blocks.

20. The palatable gastro-activated dietary fiber of claim 14, wherein the calcium alginate has a Brookfield viscosity in the range of 50 to 2000 mPas when measured at 1% polysaccharide in water containing 0.53% sodium carbonate at 20° C.

21. The palatable gastro-activated dietary fiber of claim 14, wherein the calcium alginate has a Brookfield viscosity in the range of 50 to 1000 mPas when measured at 1% polysaccharide in water containing 0.53% sodium carbonate at 20° C.

22. The palatable gastro-activated dietary fiber of claim 14, comprising 5 to 12 weight percent of calcium, based on the total weight of the gastro-activated dietary fiber.

23. The palatable gastro-activated dietary fiber of claim 14, comprising 6 to 10 weight percent of calcium, based on the total weight of the gastro-activated dietary fiber.

24. The palatable gastro-activated dietary fiber of claim 14, comprising 7 to 9 weight percent of calcium, based on the total weight of the gastro-activated dietary fiber.

25. The palatable gastro-activated dietary fiber of claim 22, wherein the calcium alginate is a powder that is free of calcium alginate fibers.

26. A gastro-activated dietary fiber comprising a calcium alginate which after exposure to an acidic pH of 2.0 for 2 hours, shows an increase in viscosity after 1 hour at pH in the range 6.9 to 7.6.

27. An ingestible composition comprising the palatable gastro-activated dietary fiber of claim 1, wherein the ingestible composition comprises from about 0.1 wt % to about 40 wt % of the gastro-activated dietary fiber.

28. An ingestible composition as claimed in claim 27, wherein the ingestible composition comprises from about 0.5 wt % to about 15 wt % of the gastro-activated dietary fiber.

29. An ingestible composition as claimed in claim 27, wherein the ingestible composition comprises from about 1 wt % to about 10 wt % of the gastro-activated dietary fiber.

30. An ingestible product comprising a gastro-activated dietary fiber as claimed in claim 1, wherein the polysaccharide salt is calcium alginate and the ingestible product comprises 5 to 12 weight percent calcium, based on the total weight of the calcium alginate.

31. An ingestible product comprising a gastro-activated dietary fiber as claimed in claim 1, wherein the polysaccharide salt is calcium alginate and the ingestible product comprises 6 to 10 weight percent calcium, based on the total weight of the calcium alginate.

32. An ingestible product comprising a gastro-activated dietary fiber as claimed in claim 1, wherein the polysaccharide salt is calcium alginate and the ingestible product comprises 7 to 9 weight percent calcium, based on the total weight of the calcium alginate.

33. The ingestible product of claim 30, further comprising an additional calcium salt.

34. The ingestible product of claim 33, wherein the calcium alginate has a density greater than or equal to 0.4 g/ml.

35. A method for providing dietary fiber to the gastro-intestinal tract comprising the steps of:
providing a palatable gastro-activated dietary fiber comprising an insoluble polysaccharide salt selected from the group of insoluble alginate salts, insoluble carrageenan salts, insoluble pectin salts and mixtures thereof,
exposing said gastro-activated dietary fiber to an acidic pH of 3 or less for at least 15 minutes; and
neutralizing said gastro-activated dietary fiber to pH within the range 4 to 7.6 to provide an increase in the viscosity of said dietary fiber.

36. A method as claimed in claim 35, wherein said exposing step occurs in a stomach of a human or animal.

37. A method as claimed in claim 36, wherein said neutralizing step occurs in a small intestine of a human or animal.

38. A method as claimed in claim 35, wherein said gastro-activated dietary fiber is provided in an ingestible composition which comprises from 0.1 to 40 weight percent of said dietary fiber, based on the total weight of the ingestible composition.

39. A method as claimed in claim 38, wherein the polysaccharide salt is calcium alginate and the ingestible product comprises 5 to 12 weight percent calcium, based on the total weight of the calcium alginate.

40. A method as claimed in claim 38, wherein the polysaccharide salt is calcium alginate and the ingestible product comprises 6 to 10 weight percent calcium, based on the total weight of the calcium alginate.

41. A method as claimed in claim 38, wherein the polysaccharide salt is calcium alginate and the ingestible product comprises 7 to 9 weight percent calcium, based on the total weight of the calcium alginate.

42. The method of claim 39, wherein the further comprising a calcium salt other than calcium alginate.

43. The method of claim 42, wherein the calcium alginate has a density greater than or equal to 0.4 g/ml.

44. The method of claim 37, wherein the insoluble polysaccharide salt has a water swelling ratio of between 1 and 15 measured after 24 hours in deionized water.

45. The method of claim 37, wherein the insoluble polysaccharide salt has a water swelling ratio of between 1 and 10 measured after 24 hours in deionized water.

46. The method of claim 37, wherein the insoluble polysaccharide salt has a water swelling ratio of between 1 and 5 measured after 24 hours in deionized water.
